(19) United States
(12) Patent Application Publication
(10) Pub. No.: US 2020/0038076 A1
(43) Pub. Date: Feb. 6, 2020

(54) METHOD AND APPARATUS FOR A CONTINUOUS COMPRESSION IMPLANT

(71) Applicant: DEPUY SYNTHES PRODUCTS, INC., Raynham, MA (US)

(72) Inventors: James A. Amis, West Chester, PA (US); Gregory S. Walters, Mount Royal, NJ (US)

(73) Assignee: DEPUY SYNTHES PRODUCTS, INC., Raynham, MA (US)

(21) Appl. No.: 16/053,016

(22) Filed: Aug. 2, 2018

(51) Int. Cl. A61B 17/80

(52) U.S. Cl. CPC A61B 17/8004; A61B 17/86; A61B 17/809

(57) ABSTRACT

An orthopedic implant includes an unconstrained shape and a constrained insertion shape. The orthopedic implant includes first and second legs and a body with a central axis and first and second ends. The body includes a first aperture therethrough positioned at the first end that receives a fixation device for securing the body at the first end with bone, bones, bone pieces, or tissue. The body further includes a second aperture therethrough positioned at the second end that receives a fixation device for securing the body at the second end with bone, bones, bone pieces, or tissue. The first leg extends from the body at a position interior of the first aperture, while the second leg extends from the body at a position interior of the second aperture.

### BACKGROUND OF THE INVENTION

### 1. Field of the Invention

The present invention relates to surgical implants suitable for use in fusing bone, bones, or bone pieces or reattaching tissue and, more particularly, but not by way of limitation, to a method and apparatus for a continuous compression implant that assists in osteosynthesis.

### 2. Description of the Related Art

Shape memory materials such as nitinol (nickel-titanium) due to their superelastic properties currently are employed in the manufacture of surgical implants designed to fuse bone, bones, or bone pieces or reattach tissue. A surgical implant manufactured from a shape memory material with superelastic properties typically includes a natural unconstrained shape. Nevertheless, the surgical implant may be deformed to an insertion shape whereby the surgical implant stores energy deliverable to a bone, bones, bone pieces, or tissue. The surgical implant when deformed to its insertion shape requires a mechanical constraint to prevent transition of the surgical implant from its insertion shape to its unconstrained shape. The surgical implant once loaded on a mechanical constraint is deliverable to a bone, bones, bone pieces, or tissue. After the surgical implant is delivered and released from the mechanical constraint, the surgical implant attempts to transition from its insertion shape to its unconstrained shape such that the surgical implant exerts a compressive force to the bone, bones, bone pieces, or tissue.

Surgical implants manufactured from a shape memory material with superelastic properties include surgical staples and surgical plates. A surgical staple typically includes a bridge with transition sections having legs extending therefrom. The surgical staple includes a natural unconstrained shape where the transition sections maintain the legs in an unconstrained position, which normally is converging. The surgical staple, however, deforms to an insertion shape where the transition sections move the legs to an open insertion position, which normally is substantially parallel. The surgical staple once deformed to its insertion shape loads on a mechanical constraint and then is deliverable to a bone, bones, bone pieces, or tissue. After the surgical staple is delivered and released from the mechanical constraint, the surgical staple attempts to transition from its insertion shape to its unconstrained shape such that the surgical staple exerts a compressive force to the bone, bones, bone pieces, or tissue. Although surgical staples operate adequately in fusing bone, bones, or bone pieces or reattaching tissue, surgical staples are prone to fixation issues whereby the surgical staples back out of the bone, bones, bone pieces, or tissue resulting in a subsequent loss of fixation.

A surgical plate typically includes a transition section and threaded apertures for receiving screws therethrough that secure the plate with bone, bones, bone pieces, or tissue. The surgical plate includes a natural unconstrained shape where the transition section maintains the plate in an unconstrained position, which normally is a closed profile with the ends of the plate residing at a first distance. The surgical plate, however, deforms to an insertion shape where the transition section moves the plate to an open insertion position, which normally is an open profile with the ends of the plate residing at a second distance that is greater than the first distance. The surgical plate once deformed to its insertion shape loads on a mechanical constraint and then is deliverable to a bone, bones, bone pieces, or tissue. In order to deliver the surgical plate, the bone, bones, bone pieces, or tissue are aligned so that holes for receiving the screws may be drilled. Once the holes are drilled, the surgical plate is secured via the screws and then released from the mechanical constraint, whereby the surgical plate attempts to transition from its insertion shape to its unconstrained shape such that the surgical plate exerts a compressive force to the bone, bones, bone pieces, or tissue. Although surgical plates operate adequately in fusing bone, bones, or bone pieces or reattaching tissue, surgical plates experience certain disadvantages. Illustratively, aligning bone, bones, bone pieces, or tissue for drilling and then maintaining the alignment during fixation of the plate is difficult such that optimal fixation is not always achieved. Moreover, a procedure involving a surgical plate often is more time consuming than other similar fixation procedures resulting in increased costs.

Accordingly, a method and apparatus for a continuous compression implant that overcomes the disadvantages currently experienced with both surgical plates and surgical staples will be beneficial in osteosynthesis and provide an improvement over surgical plates and staples.

### SUMMARY OF THE INVENTION

In accordance with the present invention, an orthopedic implant is moveable between an unconstrained shape and a constrained insertion shape. The orthopedic implant includes a body that is deformable between an unconstrained form and a constrained insertion form that transitions the orthopedic implant between its unconstrained shape and its constrained insertion shape. The body includes a central axis, first and second ends, a first aperture therethrough positioned from the central axis lengthwise along the body to a location adjacent the first end, and a second aperture therethrough positioned from the central axis lengthwise along the body to a location adjacent the second end. The first and second apertures are adapted to receive a respective fixation device that secures the body at the first and second ends with bone, bones, bone pieces, or tissue.

The orthopedic implant further includes first and second legs extending from the body. The first leg is positioned from the central axis lengthwise along the body to a location interiorly adjacent of the first aperture. Similarly, the second leg is positioned from the central axis lengthwise along the body to a location interiorly adjacent of the second aperture. The deformation of the body between its unconstrained form and its constrained insertion form moves the first and second legs between an unconstrained position and a constrained insertion position. The first and second apertures and the first and second legs are aligned lengthwise along the body such that the first and second legs are spaced apart at a first distance and the first and second apertures are spaced apart at a second distance that is greater than the first distance.

The body in its unconstrained form includes a closed profile whereby the first and second ends reside at a first distance and the first and second legs are spaced apart at a first distance. Conversely, the body in its constrained insertion form includes an open profile whereby the first and second ends reside at a second distance that is greater than the first distance and the first and second legs are spaced apart at a second distance that is greater than the first distance.

The body includes a transition section disposed at the central axis thereof that locates the body in its unconstrained form with the closed profile and further deforms to store energy and move the body from its unconstrained form to its constrained insertion form with the open profile. The orthopedic implant, which is implanted in its constrained insertion shape, delivers the energy stored in the transition section such that the body attempts to transition from its constrained insertion form to its unconstrained form. Moreover, the first and second legs attempt to move from their constrained insertion position to their unconstrained position, thereby exerting a compressive force to bone, bones, bone pieces, or tissue. The orthopedic implant, when it resides in its constrained insertion shape, may require a mechanical constraint that retains the orthopedic implant in its constrained insertion shape prior to the implantation of the orthopedic implant into bone, bones, bone pieces, or tissue.

After implantation of the orthopedic implant, the body and the first and second legs hold the bone, bones, bone pieces, or tissue in a desired alignment that permits securing of the orthopedic implant using fixation devices inserted through the first and second apertures of the body and into the bone, bones, bone pieces, or tissue without a separate holding of the bone, bones, bone pieces, or tissue. In addition, the fixation devices and their insertion respectively through the first aperture at the first end and the second aperture at the second end prevents movement or back out of the orthopedic implant from the bone, bones, bone pieces, or tissue.

Alternatively, the body includes a first transition section disposed at the first leg and a second transition section disposed at the second leg. The first and second transition sections locate the body in its unconstrained form with the first and second legs spaced apart at the first distance and further deform to store energy and move the body from its unconstrained form to its constrained insertion form with the first and second legs spaced apart at the second distance.

The body includes a bridge segment and first and second extension segments. The bridge segment includes a first end with the first leg extending therefrom and a second end with the second leg extending therefrom. The first extension segment appends from the first end of the bridge segment and includes the first aperture. The second extension segment appends from the second end of the bridge segment and includes the second aperture. The bridge segment and the first and second extension segments are in alignment with the first and second extension segments located exterior of the bridge segment. The bridge segment includes the transition section at a central axis thereof. Alternatively, the bridge segment includes the first transition section disposed at the first end thereof and the second transition section disposed at the second end thereof.

The first and second ends of the body for the orthopedic implant may include multiple discrete ends that each include an aperture and a leg extending from the body interiorly adjacent of the aperture at the discrete end. The orthopedic implant of the present invention includes bodies of different shapes that have multiple discrete ends and multiple legs in order to expand bone fusion and tissue reattachment surgeries performable using the orthopedic implant.

In a method for the orthopedic implant of the present invention, the body is deformed from its unconstrained form to its constrained insertion form, thereby transitioning the orthopedic implant from its unconstrained shape to its constrained insertion shape. This causes the first and second legs to move from to their unconstrained position to their constrained insertion. The orthopedic implant is retained in its constrained insertion shape and then implanted in bone, bones, bone pieces, or tissue. The orthopedic implant is released whereby the body attempts to transition from its constrained insertion form to its unconstrained form while the first and second legs attempt to move from their constrained insertion position to their unconstrained position. The orthopedic implant accordingly exerts a compressive force to the bone, bones, bone pieces, or tissue that holds the bone or tissue in a desired alignment. After implantation of the orthopedic implant, a first end of the body is secured with the bone, bones, bone pieces, or tissue using a first fixation device. Likewise, a second end of the body is secured with the bone, bones, bone pieces, or tissue using a second fixation device. Upon the securing of the orthopedic implant with the first and second fixation devices, the first and second legs and the first and second fixation devices are aligned lengthwise along the body such that the first and second legs are spaced apart at a first distance and the first and second fixation devices are spaced apart at a second distance that is greater than the first distance.

It is therefore an object of the present invention to provide an orthopedic implant that holds the bone, bones, bone pieces, or tissue in a desired alignment prior to insertion of fixation devices.

It is another object of the present invention to provide an orthopedic implant that includes fixation devices for preventing movement or back out of the orthopedic implant from bone, bones, bone pieces, or tissue.

Still other objects, features, and advantages of the present invention will become evident to those of ordinary skill in the art in light of the following. Also, it should be understood that the scope of this invention is intended to be broad, and any combination of any subset of the features, elements, or steps described herein is part of the intended scope of the invention.

### BRIEF DESCRIPTION OF THE DRAWINGS

FIG. 1 is an isometric view illustrating an implant according to a preferred embodiment in an unconstrained shape.

FIG. 2 is a top view thereof.

FIG. 3 is a bottom view thereof.

FIG. 4 is a front view thereof.

FIG. 5 is a side view thereof.

FIG. 6 is an isometric view illustrating the implant according to the preferred embodiment in a constrained insertion shape.

FIG. 7 is a top view thereof.

FIG. 8 is a bottom view thereof.

FIG. 9 is a front view thereof.

FIG. 10 is a side view thereof.

FIG. 11 is a front view of an implant according to an alternative of the preferred embodiment in a constrained insertion shape.

FIG. 12 is a front view of an implant according to an alternative of the preferred embodiment in an unconstrained shape.

FIG. 13 is an isometric view illustrating an example implant insertion device prior to its loading with an implant according to the preferred embodiment.

FIG. 14 is an isometric view illustrating the example implant insertion device loaded with an implant according to the preferred embodiment.

FIGS. 15-17 are isometric views illustrating insertion of the implant according to the preferred embodiment into bone, bones, or bone pieces.

FIG. 18 is an isometric view illustrating an implant according to first alternative embodiment.

FIG. 19 is an isometric view illustrating an implant according to second alternative embodiment.

### DETAILED DESCRIPTION OF THE PREFERRED EMBODIMENTS

As required, detailed embodiments of the present invention are disclosed herein; however, it is to be understood that the disclosed embodiments are merely exemplary of the invention, which may be embodied in various forms. It is further to be understood that the figures are not necessarily to scale, and some features may be exaggerated to show details of particular components or steps.

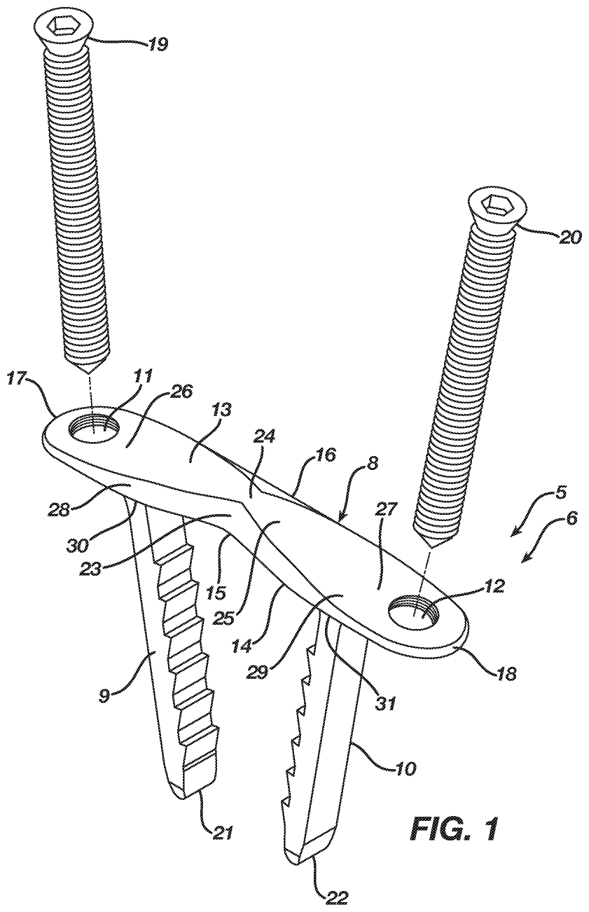

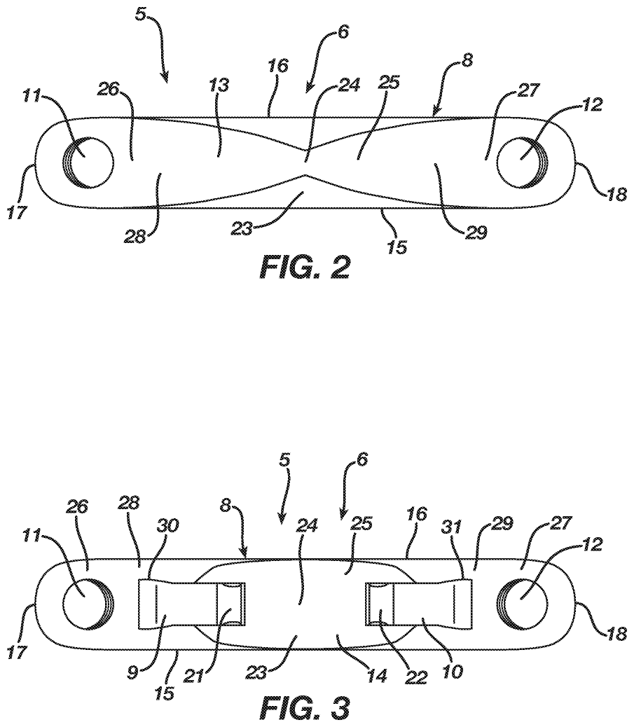

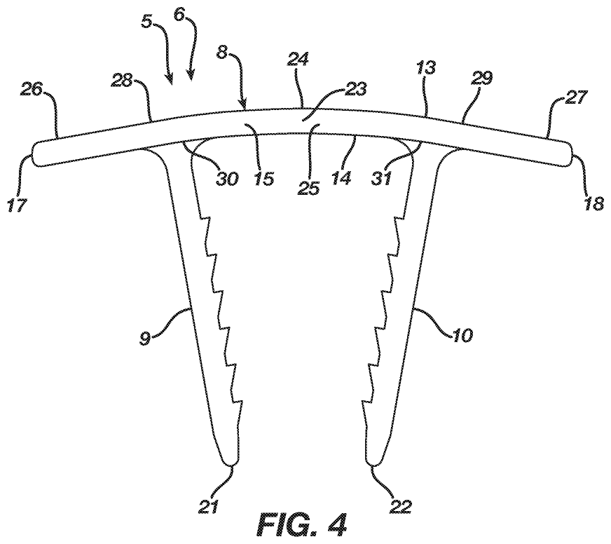

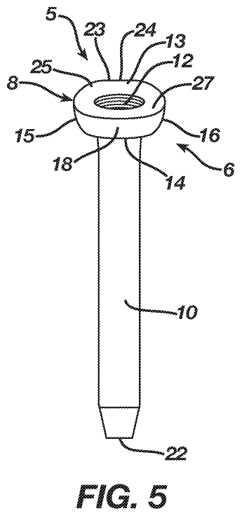

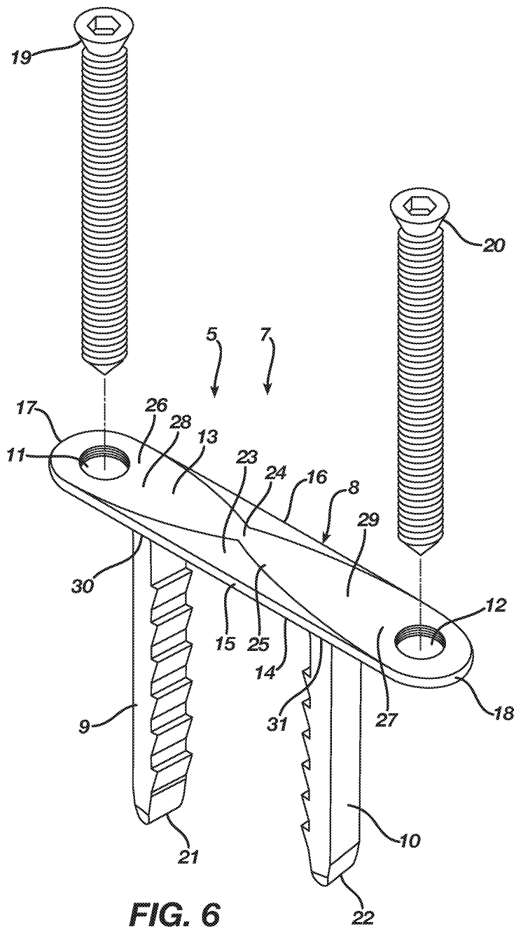

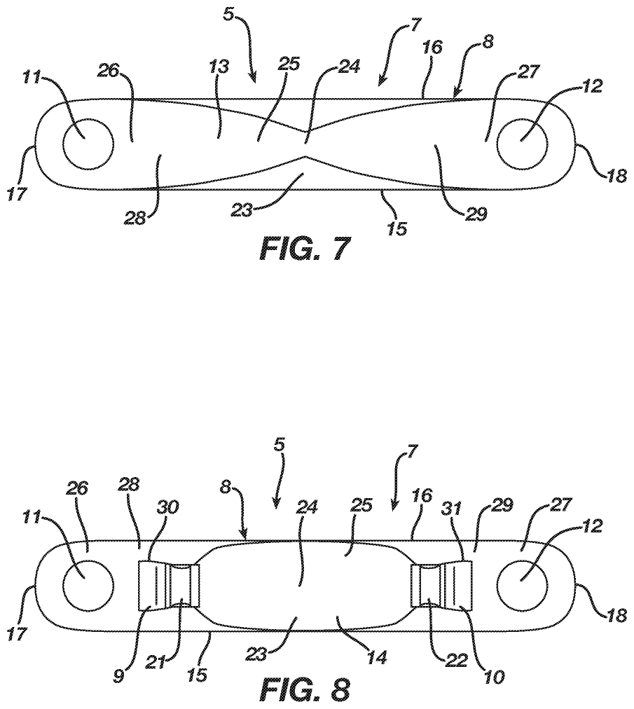

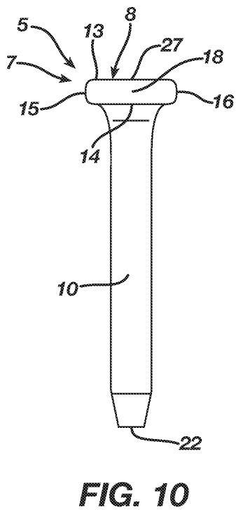

FIGS. 1-5 illustrate an orthopedic implant 5 according to a preferred embodiment in an unconstrained shape 6, whereas FIGS. 6-10 illustrate the orthopedic implant 5 in a constrained insertion shape 7. The implant 5 in the preferred embodiment accordingly is a continuous compression implant that assists in fusing bone, bones, or bone pieces or reattaching tissue. The implant 5 may be manufactured from any elastic material suitable for orthopedic use, such as a shape memory material (e.g., Nitinol). In the preferred embodiment, the implant 5 includes a body 8 having a central axis 24 and legs 9 and 10 extending from the body 8. Each leg 9 and 10, which has a respective tip 21 and 22, may include barbs thereon that improve the pull-out resistance of the implant 5.

The body 8 in the preferred embodiment is three-dimensional in form having a length, width, and height, and, in particular, the body 8 is plate-shaped and includes an upper surface 13 and a lower surface 14 with first and second sides 15 and 16 and first and second ends 17 and 18 therebetween. The body 8 is tapered to present a non-uniform cross-sectional thickness between the upper and lower surfaces 13 and 14 in order to provide strength to the body 8 while lowering its profile. Although the body 8 is tapered in the preferred embodiment, one of ordinary skill in the art will recognize that the body 8 may include a uniform cross-sectional thickness between the upper and lower surfaces 13 and 14.

The body 8 includes a first aperture 11 therethrough that, in the preferred embodiment, is threaded and receives therethrough a fixation device 19, such as a non-locking or locking bone screw. The first aperture 11 is positioned from the central axis 24 lengthwise along the body 8 to a location adjacent the first end 17. Likewise, the body 8 includes a second aperture 12 therethrough that, in the preferred embodiment, is threaded and receives therethrough a fixation device 20, such as a non-locking or locking bone screw. The second aperture 12 is positioned from the central axis 24 lengthwise along the body 8 to a location adjacent the second end 18. The leg 9 extends from the lower surface 15 of the body 8 and further is positioned from the central axis 24 lengthwise along the body 8 to a location interiorly adjacent of the first aperture 11. Similarly, the leg 10 extends from the lower surface 15 of the body 8 and further is positioned from the central axis 24 lengthwise along the body 8 to a location interiorly adjacent of the second aperture 12. The first and second apertures 11 and 12 and the legs 9 and 10 accordingly are aligned lengthwise along the body 8 such that the legs 9 and 10 are spaced apart at a first distance and the first and second apertures 11 and 12 are spaced apart at a second distance that is greater than the first distance.

The body 8 in the preferred embodiment includes a bridge segment 25 terminating at the legs 9 and 10, an extension segment 26 appended from an end 28 of the bridge segment 25, and an extension segment 27 appended from an end 29 of the bridge segment 25. The leg 9 extends from the end 28 of the bridge segment 25, whereas the leg 10 extends from the end 29 of the bridge segment 25. The bridge segment 25 facilitates transition of the implant 5 between its unconstrained shape 6 and its constrained insertion shape 7. The extension segment 26 extends lengthwise along the body 8 exterior to the end 28 of the bridge segment 25 and includes the first aperture 11 therethrough in order to receive the fixation device 19 such that the extension segment 26 assists in maintaining the implant 5 secured with bone, bones, bone pieces, or tissue. Likewise, the extension segment 27 extends lengthwise along the body 8 exterior to the end 29 of the bridge segment 25 and includes the first aperture 12 therethrough in order to receive the fixation device 20 such that the extension segment 27 assists in maintaining the implant 5 secured with bone, bones, bone pieces, or tissue. The bridge segment 25 and the extension segments 26 and 27 accordingly are in alignment with the extension segments 26 and 27 located exterior of the bridge segment 25.

The body 8 in the preferred embodiment includes a transition section 23 disposed at the central axis 24 thereof, and, in particular, the transition section 23 is centered in the bridge segment 25. The natural shape of the implant 5, as illustrated in FIGS. 1-5, is its unconstrained shape 6 where the transition section 23 locates the body 8 in an unconstrained form, which, in the preferred embodiment, is a closed or angular profile whereby the first and second ends 17 and 18 reside at a first distance and the legs 9 and 10 reside in an unconstrained position, which is convergent whereby the legs 9 and 10 are spaced apart at a first distance. Nevertheless, as illustrated in FIGS. 6-10, the implant 5 is deformable under the action of superelasticity or shape memory to a constrained insertion shape 7 where the transition section 23 deforms to store energy while also moving the body 8 from its unconstrained form to a constrained insertion form which, in the preferred embodiment, is an open or substantially linear profile whereby the first and second ends 17 and 18 reside at a second distance that is greater than the first distance and the legs 9 and 10 reside in a constrained insertion position, which is substantially parallel whereby the legs 9 and 10 are spaced apart at a second distance that is greater than the first distance. Since the constrained insertion shape 7 is not the natural shape of the implant 5, the transition section 23 must be mechanically constrained or the implant 5 must be chilled until it reaches its martensite phase whereby the transition section 23 once deformed maintains the body 8 in its constrained insertion form. Release of a mechanical constraint or the heating of the implant 5 to its austenite phase results in the implant 5 delivering the energy stored in the transition section 23 such that the body 8 attempts to transition from its constrained insertion form to its unconstrained form resulting in the legs 9 and 10 attempting to move from their constrained insertion position to their unconstrained position thereby exerting a compressive force to bone, bones, bone pieces, or tissue after implantation.

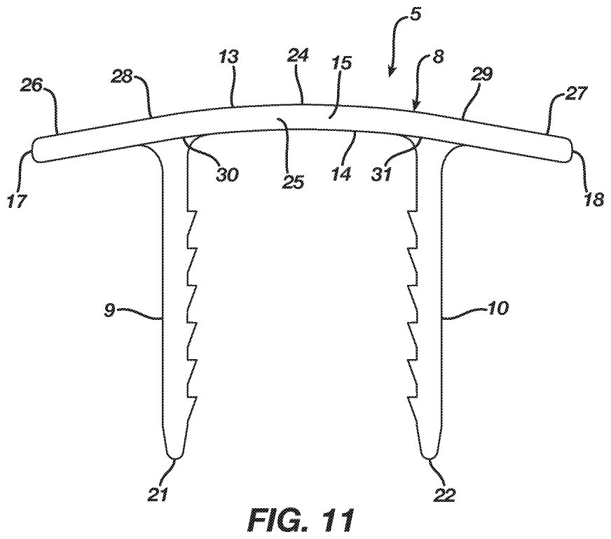

Alternatively, the body 8 in the preferred embodiment may include transition sections 30 and 31 located respectively where the legs 9 and 10 extend from the body 8, and, in particular, the transition sections 30 and 31 may reside respectively at the ends 28 and 29 of the bridge segment 25. The natural shape of the implant 5, as illustrated in FIGS. 1-5, is its unconstrained shape 6 where the transition sections 30 and 31 locate the body 8 in an unconstrained form whereby the legs 9 and 10 reside in an unconstrained position, which, in the preferred embodiment, is convergent whereby the legs 9 and 10 are spaced apart at a first distance. Nevertheless, as illustrated in FIG. 11, the implant 5 is deformable under the action of superelasticity or shape memory to a constrained insertion shape 7 where the transition sections 30 and 31 deform to store energy while also moving the body 8 from its unconstrained form to a constrained insertion form whereby the legs 9 and 10 reside in a constrained insertion position, which, in the preferred embodiment, is substantially parallel whereby the legs 9 and 10 are spaced apart at a second distance that is greater than the first distance. Since the constrained insertion shape 7 is not the natural shape of the implant 5, the transition section 23 must be mechanically constrained or the implant 5 must be chilled until it reaches its martensite phase whereby the transition sections 30 and 31 once deformed maintain the body 8 in its constrained insertion form. Release of a mechanical constraint or the heating of the implant 5 to its austenite phase results in the implant 5 delivering the energy stored in the transition sections 30 and 31 such that the body 8 attempts to transition from its constrained insertion form to its unconstrained form whereby the legs 9 and 10 attempt to move from their constrained insertion position to their unconstrained position thereby exerting a compressive force to bone, bones, bone pieces, or tissue after implantation.

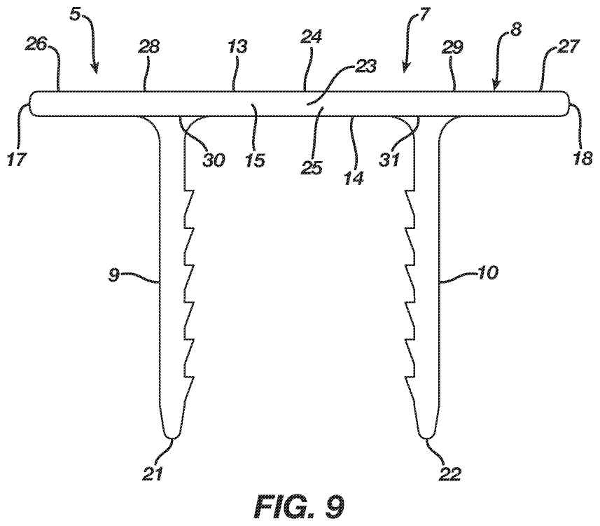

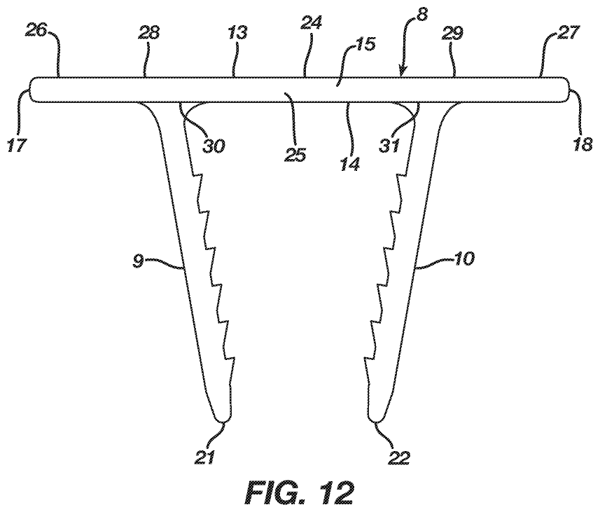

Although the preferred embodiment of the implant 5 includes either the transition section 23 or the transition sections 30 and 31 to produce deformation thereof, one of ordinary skill in the art will recognize that the body 8 of the implant 5 may include both the transition section 23 and the transition sections 30 and 31 to produce deformation thereof. Moreover, while the body 8 includes an angular profile in the unconstrained shape 6 of the implant 5, it should be understood by one of ordinary skill in the art that, when the body 8 incorporates the transition sections 30 and 31, the body 8 as illustrated in FIG. 12 may include a substantially linear profile in an unconstrained shape of the implant 5. Furthermore, the body 8 maintains its substantially linear profile once the implant 5 deforms to a constrained insertion shape, as shown in FIG. 9 which may be considered an illustrative example thereof.

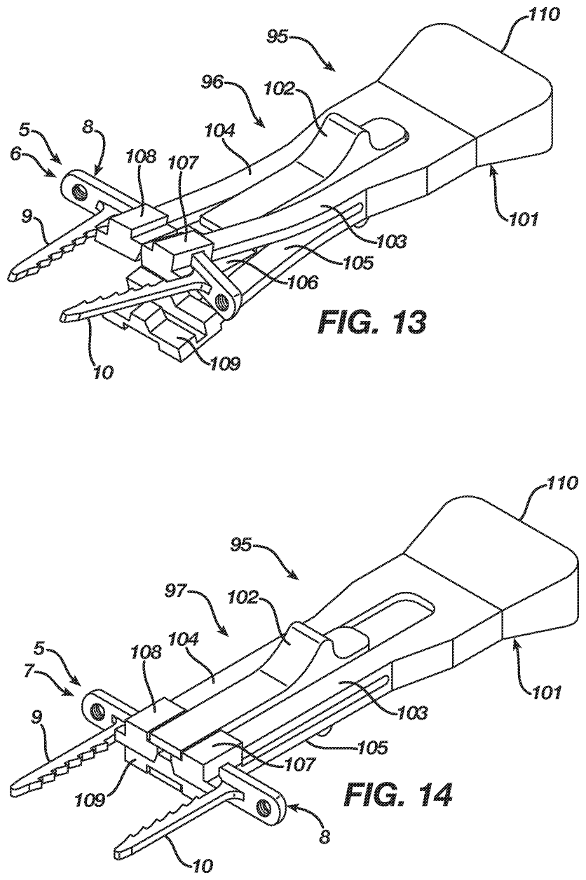

FIGS. 13 and 14 illustrate an implant insertion device 95, which is presented herein as an example of a mechanical constraint suitable to engage the implant 5 and maintain the implant 5 in its constrained insertion shape 7. FIG. 13 illustrates the implant insertion device 95 prior to its loading with the implant 5, whereas FIG. 14 illustrates the implant insertion device 95 loaded with the implant 5. Implant insertion devices suitable to maintain the implant 5 in its constrained insertion shape 7, such as the exemplary implant insertion device 95, are available from DePuy Synthes Products, Inc., 325 Paramount Drive, Rayham, Mass. 02767.

The implant insertion device 95 resides in either an implant disengagement position 96 as illustrated in FIG. 13 or an implant engagement position 97 as illustrated in FIG. 14 and is movable therebetween. In the implant disengagement position 96, the implant 5 slips into or out of the implant insertion device 95 with no obstruction. In the implant engagement position 97, the implant insertion device 95 engages the implant 5 and maintains the implant 5 in its constrained insertion shape 7. In addition, the implant insertion device 95 allows a surgeon to manipulate the implant 5 and insert the implant 5 into bone, bones, bone pieces, or tissue that require fixating.

The implant insertion device 95 includes a body 101 and a slider 102. The body 101 is configured to accept the slider 102 such that the slider 102 moves along the body 101 between an unlocked and a locked position. The body 101 includes a handle 110 and arms 103-106 extending from the handle 110. The handle 110 allows manipulation of the implant insertion device 95 and delivery of the implant 5 into bone, bones, bone pieces, or tissue. The first arm 105 terminates in a first jaw 109 configured to engage the implant 5. Likewise, the second arm 106 terminates in a second jaw 110 configured to engage the implant 5. The third and fourth arms collectively terminate in a third jaw 111 configured to engage the implant 5. The jaws 107-109 move between a disengaged position that releases the implant 5 and an engaged position whereby the jaws 107-109 engage the implant 5 and maintain the implant 5 in its constrained insertion shape 7. The jaws 107-109 are configured to accept the slider 102 such that, when the slider 102 resides in its locked position, the slider 102 holds the jaws 108-109 in their engaged position. Conversely, when the slider 102 resides in its unlocked position, the slider 102 releases the jaws 107-109 such that the jaws 107-109 move to their disengaged position.

In a first method of receiving the implant 5, the implant insertion device 95 begins in its implant disengagement position 96 wherein the jaws 107-109 reside in their disengaged position. The implant 5 is mechanically deformed from its unconstrained shape 6 to its constrained insertion shape 7 such that the implant 5 stores mechanical energy. Once deformed, the implant 5 inserts within the jaws 107 and 108; in particular, a portion of the body 8, which, in the preferred embodiment, is the bridge segment 25, inserts within the jaws 107 and 108. After insertion of the implant 5, the jaws 107-109 are moved from their disengaged position to their engaged position, which, in the preferred embodiment, entails movement of the jaws 107 and 108 downward and the jaw 109 upward until the jaws 107 and 108 abut the jaw 109. Abutting the jaws 107 and 108 with the jaw 109 results in the jaw 109 receiving therein the implant 5 and, in particular, a portion of the body 8, which, in the preferred embodiment, is the bridge segment 25 such that the jaws 107-109 engage the body 8 at the bridge segment 25 thereby maintaining the implant 5 in its constrained insertion shape 7. Abutting the jaws 107 and 108 with the jaw 109 further results in the jaw 109 urging the jaws 107 and 108 to engage the implant 5 and, in particular, the legs 9 and 10 such that the jaws 107 and 108 contact the legs 9 and 10 thereby maintaining the implant 5 in its constrained insertion shape 7. With the jaws 107-109 now moved to their engaged position, the slider 102 is progressed from its unlocked to its locked position where the slider 102 holds the jaws 108-109 in their engaged position thereby clamping the implant 5 between the jaws 107-109 such that implant 5 remains loaded on the implant insertion device 95 in its constrained insertion shape 7 with mechanical energy stored therein.

While the implant 5 may be mechanically deformed from its unconstrained shape 6 to its constrained insertion shape 7 before loading on the implant insertion device 95, in a second method, the implant 5 may be loaded on the implant insertion device 95 in its unconstrained shape 6 and then mechanically deformed to its constrained insertion shape 7 by the implant insertion device 95. The implant 5 inserts in its unconstrained shape 6 within the jaws 107 and 108; in particular, a portion of the body 8, which, in the preferred embodiment, is the bridge segment 25, inserts within the jaws 107 and 108. After insertion of the implant 5, the jaws 107-109 are moved from their disengaged position to their engaged position, which, in the preferred embodiment, entails movement of the jaws 107 and 108 downward and the jaw 109 upward until the jaws 107 and 108 abut the jaw 109. Abutting the jaws 107 and 108 with the jaw 109 results in the jaw 109 receiving therein the implant 5 and, in particular, a portion of the body 8, which, in the preferred embodiment, is the bridge segment 25 such that the jaws 107-109 engage the body 8 at the bridge segment 25 whereby the jaws 107-109 impart a force to the implant 5 such that the implant 5 mechanically deforms from its unconstrained shape 6 to its constrained insertion shape 7 further whereby the jaws 107-109 maintain the implant 5 in its constrained insertion shape 7. Abutting the jaws 107 and 108 with the jaw 109 further results in the jaw 109 urging the jaws 107 and 108 to engage the implant 5 and, in particular, the legs 9 and 10 such that the jaws 107 and 108 contact the legs 9 and 10 whereby the jaws 107 and 108 impart a force to the implant 5 such that the implant 5 mechanically deforms from its unconstrained shape 6 to its constrained insertion shape 7 further whereby the jaws 107 and 108 maintain the implant 5 in its constrained insertion shape 7. With the jaws 107-109 now moved to their engaged position, the slider 102 is progressed from its unlocked to its locked position where the slider 102 holds the jaws 108-109 in their engaged position thereby clamping the implant 5 between the jaws 107-109 such that implant 5 remains loaded on the implant insertion device 95 in its constrained insertion shape 7 with mechanical energy stored therein. Although not necessary, the implant 5 may be cooled prior to loading on the implant insertion device 95 in order to place it in a martensitic state and aid in movement of the implant 5 from its unconstrained shape 6 to its constrained insertion shape 7.

The implant insertion device 95 and its loading with the implant 5 has been shown in order to provide an example mechanical constraint. Nevertheless, one of ordinary skill in the art will recognize that any mechanical device, such as forceps, suitable to engage the implant 5 and maintain the implant 5 in its constrained insertion shape 7 may be employed.

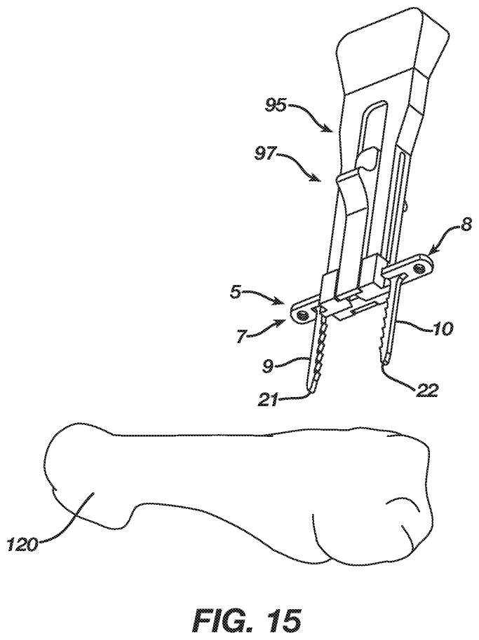

FIG. 15 illustrates the implant insertion device 95 with the implant 5 loaded thereon whereby the implant insertion device 95 retains the implant 5 in its constrained insertion shape 7 such that the implant 5 is ready for implantation in bone, bones, bone pieces, or tissue, and, in particular, into a bone 120 which is presented herein as an example. A surgeon aligns the bone 120 and then drills holes therein at a desired location and spacing for insertion of the legs 9 and 10 into the bone 120 when the implant 5 resides in its constrained insertion shape 7. The surgeon next utilizes the implant insertion device 95 to position the tips 21 and 22 of the legs 9 and 10 at the pre-drilled holes and then insert the legs 9 and 10 into the bone 120 via the pre-drilled holes. Alternatively, the surgeon may align the bone 120 followed by the use of the implant insertion device 95 to impact the legs 9 and 10 into the bone 120 at a desired location.

Once the implant 5 inserts into the bone 120, the implant 5 is ready for removal from the implant insertion device 95. To remove the implant 5 from the implant insertion device 95, the surgeon progresses the slider 102 from its locked position to its unlocked position resulting in the slider 102 releasing the jaws 107-109. The released jaws 107-109 travel from their engaged position to their disengaged position such that the implant insertion device 95 transitions from its implant engagement position 97 to its implant disengagement position 96 whereby the implant 5 disengages from the implant insertion device 95 without obstruction. In particular, the jaws 107 and 108 move away from the jaw 109 until the jaws 107 and 108 no longer abut the jaw 109. As a consequence, the jaws 107-109 release the implant 5 thereby allowing the surgeon to remove the implant insertion device 95 from the implant 5 thereby leaving the implant 5 within the bone 120. After disengaging the implant insertion device 95 from the implant 5, the surgeon tamps the implant 5 in abutting relationship with bone 120.

With the implant 5 released from the implant insertion device 95 and inserted into the bone 120, the implant 5 attempts to transition from its constrained insertion shape 7 to its unconstrained shape 6 such that the implant 5 through its continuous compression of the bone 120 remains implanted in the bone 120 thereby holding the bone 120 in a desired alignment and assisting in the fusing thereof. In particular, the body 8 attempts to transition from its unconstrained form to its constrained insertion form while the legs 9 and 10 attempt to move from their constrained insertion position to their unconstrained position.

The implant 5, due to its continuous compression of the bone 120 and its securing of the bone 120 in a desired alignment, permits a surgeon to further secure the implant 5 with the bone 120 using the fixation devices 19 and 20 without the normal necessity of having to hold the bone 120 in the desired alignment during the insertion of the fixation devices 19 and 20. The implant 5, accordingly, eliminates a difficult bone holding step experienced during the insertion of the fixation devices 19 and 20, which improves fixation alignment of the bone 120 and thus its ultimate fusing while also reducing the time required to perform the surgical procedure.

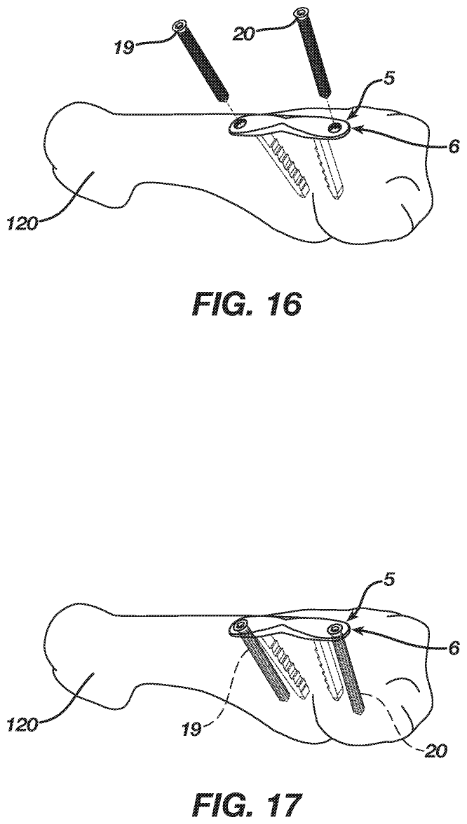

FIGS. 16 and 17 illustrate further securing of the implant 5 with bone, bones, bone pieces, or tissue, and, in particular, with the bone 120, which is presented herein as an example, employing the fixation devices 19 and 20. In the example presented in FIGS. 16 and 17, the fixation devices 19 and 20 are screws that may be any suitable screw such as a non-locking or locking bone screw including a self-tapping bone screw. After the surgeon implants the implant 5 into the bone 120, the surgeon then inserts the fixation devices 19 and 20 through the implant 5 and into the bone 120. In one insertion method, the surgeon inserts a first self-tapping bone screw through the first aperture 11 of the body 8 and into the bone 120 and a second self-tapping bone screw through the second aperture 12 of the body 8 and into the bone 120. Alternatively, the surgeon may drill holes into the bone 120 at the first and second apertures 11 and 12 using the first and second apertures as drill guides or employing a drill guide located at the first and second apertures. The surgeon then inserts a first bone screw through the first aperture 11 of the body 8 and into the pre-drilled hole in the bone 120 and a second bone screw through the second aperture 12 of the body 8 and into the pre-drilled hole in bone 120. In the preferred embodiment, any bone screws inserted into the bone 120 engage the body 8 via threads at the first and second apertures 11 and 12. The implant 5, accordingly, improves the fixation and the ultimate fusing of the bone 120 due to its incorporation of the bridge segment 25 and, in particular, the extension segments 26 and 27 that respectively receive the fixation devices 19 and 20, which assist in preventing fixation loss due to movement of the implant 5 or its backing out from the bone 120.

The implant 5 in view of the foregoing overcomes disadvantages currently experienced with both surgical plates and surgical staples. In particular, the implant 5 includes an unconstrained shape 6 and a constrained insertion shape 7 such that after insertion the implant 5 delivers continuous compression via its body 8 and its legs 9 and 10. As a result, the implant 5 secures the bone 120 in a desired alignment that eliminates a difficult bone holding step experienced during the insertion of fixation. The implant 5, accordingly, improves fixation alignment of bone, bones, bone pieces, or tissue and thus their ultimate fusing while also reducing the time required to perform the surgical procedure. Furthermore, the implant 5 includes the body 8 with first and second apertures 11 and 12 located at respective ends 17 and 18 thereof that permit the securing of the implant 5 with bone, bones, bone pieces, or tissue using fixation devices such as bone screws. As a result, the implant 5 exhibits enhanced movement or back out prevention such that the implant 5 improves the fixation and the ultimate fusing of bone, bones, bone pieces, or tissue.

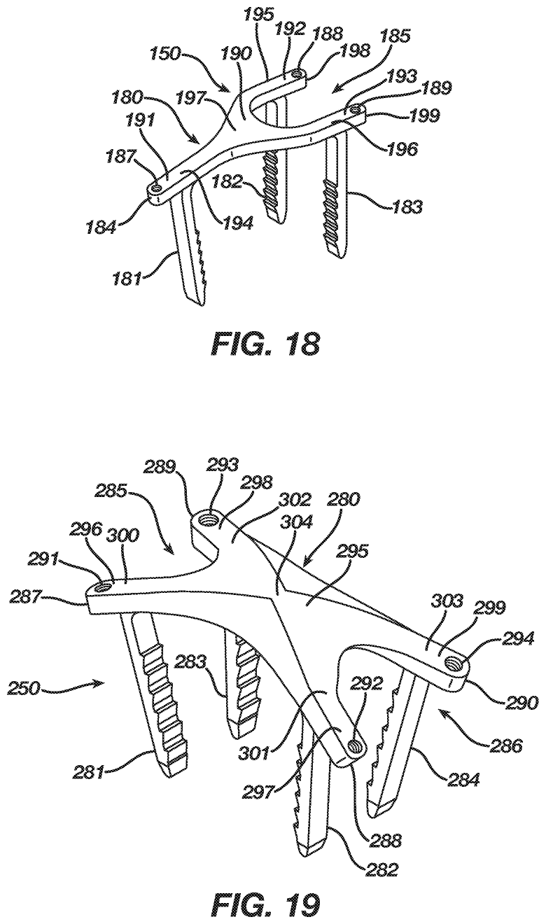

FIG. 18 illustrates an orthopedic implant 150 according to a first alternative embodiment in an unconstrained shape. The implant 150 includes the unconstrained shape and also a constrained insertion shape whereby the implant 150 provides continuous compression that assists in fusing bone, bones, or bone pieces or reattaching tissue. The implant 150 may be manufactured from any elastic material suitable for orthopedic use, such as a shape memory material (e.g., Nitinol). The implant 150 in the first alternative embodiment includes a shape that differs from the implant 5 of the preferred embodiment in order to expand bone fusion and tissue reattachment surgeries performable using an implant according to the present invention. Nevertheless, the design concept and principle of operation for the implant 150 in the first alternative embodiment is substantially identical to that of the implant 5 according to the preferred embodiment.

The implant 150 in the first alternative embodiment includes a body 180 that differs in shape from the body 8 of the implant 5 and legs 181-183 that differ in number from the legs 9 and 10 of the implant 5. The body 180 in the first alternative embodiment is three-dimensional in form having a length, width, and height, and, in particular, the body 180 is plate-shaped with a Y configuration including a first end 184 and a second end 185 comprised of discrete ends 198 and 199. The body 180 includes first, second, and third apertures 187-189 positioned from a central axis lengthwise along the body 180 to locations adjacent respective first end 184 and respective discrete ends 198 and 199. The legs 181-183 extend from a lower surface of the body 180 and further are positioned from the central axis lengthwise along the body 180 to a location interiorly adjacent respectively of the first, second, and third apertures 187-189. The body 180 further includes a bridge segment 190 terminating respectively at the legs 181-183 and extension segments 191-193 appended respectively from ends 194-196 of the bridge segment 190.

Although the body 180 of the implant 150 differs in shape from the body 8 of the implant 5 and includes three legs 181-183, the body 180 is substantially identical to the body 8 in that the body 180 includes a transition section 197 that locates the body 180 in an unconstrained form, which, in the preferred embodiment, is a closed or angular profile whereby the first end 184 and the second end 185 reside at a first distance and the legs 181-183 reside in an unconstrained position, which is convergent. Nevertheless, the implant 150 is deformable under the action of superelasticity or shape memory to its constrained insertion shape where the transition section 197 deforms to store energy while also moving the body 180 from its unconstrained form to a constrained insertion form which, in the preferred embodiment, is an open or substantially linear profile whereby the first end 184 and the second end 185 reside at a second distance that is greater than the first distance and the legs 181-183 reside in a constrained insertion position, which is substantially parallel. Alternatively, the body 180 may include transition sections located respectively where the legs 181-183 extend from the body 180.

In light of the implant 150 according to the first alternative embodiment being substantially identical in design concept and principle of operation to that of the implant 5 according to the preferred embodiment, the method of implanting the implant 150 into bone, bones, bone pieces, or tissue is substantially identical to that of the implant 5. By way of example, the implant 150 is mechanically constrained with a suitable implant insertion device that assists a surgeon in implanting the implant 150 in its constrained insertion shape. After implantation and release of the implant insertion device, the implant 180 delivers energy stored in the transition section 197 such that the body 180 attempts to transition from its constrained insertion form to its unconstrained form resulting in the legs 181-183 attempting to move from their constrained insertion position to their unconstrained position thereby exerting a compressive force to bone, bones, bone pieces, or tissue.

FIG. 19 illustrates an orthopedic implant 250 according to a second alternative embodiment in an unconstrained shape. The implant 250 includes the unconstrained shape and also a constrained insertion shape whereby the implant 250 provides continuous compression that assists in fusing bone, bones, or bone pieces or reattaching tissue. The implant 250 may be manufactured from any elastic material suitable for orthopedic use, such as a shape memory material (e.g., Nitinol). The implant 250 in the second alternative embodiment includes a shape that differs from the implant 5 of the preferred embodiment in order to expand bone fusion and tissue reattachment surgeries performable using an implant according to the present invention. Nevertheless, the design concept and principle of operation for the implant 250 in the second alternative embodiment is substantially identical to that of the implant 5 according to the preferred embodiment.

The implant 250 in the second alternative embodiment includes a body 280 that differs in shape from the body 8 of the implant 5 and legs 281-284 that differ in number from the legs 9 and 10 of the implant 5. The body 280 in the second alternative embodiment is three-dimensional in form having a length, width, and height, and, in particular, the body 280 is plate-shaped with an X configuration including a first end 285 and a second end 286 comprised of discrete ends 287-290. The body 280 includes first, second, third, and fourth apertures 291-294 positioned from a central axis lengthwise along the body 280 to locations adjacent respective discrete ends 287-290. The legs 281-284 extend from a lower surface of the body 280 and further are positioned from the central axis lengthwise along the body 280 to a location interiorly adjacent respectively of the first, second, third, and fourth apertures 291-294. The body 280 further includes a bridge segment 295 terminating respectively at the legs 281-284 and extension segments 296-299 appended respectively from ends 300-303 of the bridge segment 295.

Although the body 280 of the implant 250 differs in shape from the body 8 of the implant 5 and includes four legs 281-284, the body 280 is substantially identical to the body 8 in that the body 280 includes a transition section 304 that locates the body 280 in an unconstrained form, which, in the preferred embodiment, is a closed or angular profile whereby the first end 285 and the second end 286 reside at a first distance and the legs 281-284 reside in an unconstrained position, which is convergent. Nevertheless, the implant 250 is deformable under the action of superelasticity or shape memory to its constrained insertion shape where the transition section 304 deforms to store energy while also moving the body 280 from its unconstrained form to a constrained insertion form which, in the preferred embodiment, is an open or substantially linear profile whereby the first end 285 and the second end 286 reside at a second distance that is greater than the first distance and the legs 281-284 reside in a constrained insertion position, which is substantially parallel. Alternatively, the body 280 may include transition sections located respectively where the legs 281-284 extend from the body 280.

In light of the implant 250 according to the second alternative embodiment being substantially identical in design concept and principle of operation to that of the implant 5 according to the preferred embodiment, the method of implanting the implant 250 into bone, bones, bone pieces, or tissue is substantially identical to that of the implant 5. By way of example, the implant 250 is mechanically constrained with a suitable implant insertion device that assists a surgeon in implanting the implant 250 in its constrained insertion shape. After implantation and release of the implant insertion device, the implant 280 delivers energy stored in the transition section 304 such that the body 280 attempts to transition from its constrained insertion form to its unconstrained form resulting in the legs 281-284 attempting to move from their constrained insertion position to their unconstrained position thereby exerting a compressive force to bone, bones, bone pieces, or tissue.

In view of the foregoing embodiments illustrating an orthopedic implant according to the present invention, it should be understood that a continuous compression orthopedic implant will fall within the scope of the present invention regardless of its body shape and number of legs provided the implant body receives fixation devices at ends thereof. Moreover, although the present invention has been described in terms of the foregoing embodiments, such description has been for exemplary purposes only and, as will be apparent to those of ordinary skill in the art, many alternatives, equivalents, and variations of varying degrees will fall within the scope of the present invention. That scope, accordingly, is not to be limited in any respect by the foregoing detailed description; rather, it is defined only by the claims that follow.

1. An orthopedic implant moveable between an unconstrained shape and a constrained insertion shape, comprising:
a body with a central axis and first and second ends, the body being deformable between an unconstrained form and a constrained insertion form that transitions the orthopedic implant between its unconstrained shape and its constrained insertion shape, the body, including:
a first aperture therethrough positioned from the central axis lengthwise along the body to a location adjacent the first end, wherein the first aperture is adapted to receive a fixation device that secures the body at the first end with a bone or tissue, and
a second aperture therethrough positioned from the central axis lengthwise along the body to a location adjacent the second end, wherein the second aperture is adapted to receive a fixation device that secures the body at the second end with the bone or tissue;
a first leg extending from the body, wherein the first leg is positioned from the central axis lengthwise along the body to a location interiorly adjacent of the first aperture; and
a second leg extending from the body, wherein the second leg is positioned from the central axis lengthwise along the body to a location interiorly adjacent of the second aperture, further wherein deformation of the body between its unconstrained form and its constrained insertion form moves the first and second legs between an unconstrained position and a constrained insertion position.

2. The orthopedic implant according to claim 1, wherein the first and second apertures and the first and second legs are aligned lengthwise along the body such that the first and second legs are spaced apart at a first distance and the first and second apertures are spaced apart at a second distance that is greater than the first distance.

3. The orthopedic implant according to claim 1, wherein the body in its unconstrained form includes a closed profile whereby the first and second ends reside at a first distance and the first and second legs are spaced apart at a first distance, further wherein the body in its constrained insertion form includes an open profile whereby the first and second ends reside at a second distance that is greater than the first distance and the first and second legs are spaced apart at a second distance that is greater than the first distance.

4. The orthopedic implant according to claim 3, wherein the body includes a transition section disposed at the central axis thereof, whereby the transition section locates the body in its unconstrained form with the closed profile, further whereby the transition section deforms to store energy and move the body from its unconstrained form to its constrained insertion form with the open profile.

5. The orthopedic implant according to claim 4, wherein the orthopedic implant is implanted in its constrained insertion shape, further wherein, after implantation, the orthopedic implant delivers the energy stored in the transition section such that the body attempts to transition from its constrained insertion form to its unconstrained form resulting in the first and second legs attempting to move from their constrained insertion position to their unconstrained position, thereby exerting a compressive force to bone or tissue.

6. The orthopedic implant according to claim 5, wherein the body and the first and second legs of the orthopedic implant after implantation thereof hold the bone or tissue in a desired alignment such that a securing of the orthopedic implant using fixation devices inserted through the first and second apertures of the body and into the bone or tissue is performable without a separate holding of the bone or tissue.

7. The orthopedic implant according to claim 6, wherein the body and the first aperture thereof located at the first end and the second aperture thereof located at the second end permit a securing of the orthopedic implant with the bone or tissue using fixation devices that prevent movement or back out of the orthopedic implant from the bone or tissue.

8. The orthopedic implant according to claim 1, wherein the body in its unconstrained form includes the first and second legs spaced apart at a first distance, further wherein the body in its constrained insertion form includes the first and second legs spaced apart at a second distance that is greater than the first distance.

9. The orthopedic implant according to claim 8, wherein the body includes a first transition section disposed at the first leg and a second transition section disposed at the second leg, whereby the first and second transition sections locate the body in its unconstrained form with the first and second legs spaced apart at the first distance, further whereby the first and second transition sections deform to store energy and move the body from its unconstrained form to its constrained insertion form with the first and second legs spaced apart at the second distance.

10. The orthopedic implant according to claim 9, wherein the orthopedic implant is implanted in its constrained insertion shape, further wherein, after implantation, the orthopedic implant delivers the energy stored in the transition sections such that the body attempts to transition from its constrained insertion form to its unconstrained form resulting in the first and second legs attempting to move from their constrained insertion position to their unconstrained position, thereby exerting a compressive force to bone or tissue.

11. The orthopedic implant according to claim 10, wherein the body and the first and second legs of the orthopedic implant after implantation thereof hold the bone or tissue in a desired alignment such that a securing of the orthopedic implant using fixation devices inserted through the first and second apertures of the body and into the bone or tissue is performable without a separate holding of the bone or tissue.

12. The orthopedic implant according to claim 11, wherein the body and the first aperture thereof located at the first end and the second aperture thereof located at the second end permit a securing of the orthopedic implant with the bone or tissue using fixation devices that prevent movement or back out of the orthopedic implant from the bone or tissue.

13. The orthopedic implant according to claim 4, the body, further including:
a bridge segment having a first end with the first leg extending therefrom and a second end with the second leg extending therefrom;
a first extension segment appended from the first end of the bridge segment, wherein the first extension segment includes the first aperture; and
a second extension segment appended from the second end of the bridge segment, wherein the second extension segment includes the second aperture.

14. The orthopedic implant according to claim 13, wherein the bridge segment and the first and second extension segments are in alignment with the first and second extension segments located exterior of the bridge segment.

15. The orthopedic implant according to claim 13, wherein the bridge segment includes the transition section at a central axis thereof.

16. The orthopedic implant according to claim 13, wherein the bridge segment includes the first transition section disposed at the first end thereof and the second transition section disposed at the second end thereof.

17. The orthopedic implant according to claim 1, wherein the orthopedic implant, when it resides in its constrained insertion shape, requires a mechanical constraint that retains the orthopedic implant in its constrained insertion shape, further wherein, after implantation and release of the mechanical constraint, the orthopedic implant attempts to transition from its constrained insertion shape to its unconstrained shape, thereby exerting a compressive force to bone or tissue.

18. The orthopedic implant according to claim 17, wherein the body and the first and second legs of the orthopedic implant after implantation and release of the mechanical constraint hold the bone or tissue in a desired alignment such that a securing of the orthopedic implant using fixation devices inserted through the first and second apertures of the body and into the bone or tissue is performable without a separate holding of the bone or tissue.

19. The orthopedic implant according to claim 19, wherein the body and the first aperture thereof located at the first end and the second aperture thereof located at the second end permit a securing of the orthopedic implant with the bone or tissue using fixation devices that prevent movement or back out of the orthopedic implant from the bone or tissue.

20. The orthopedic implant according to claim 1, wherein:
the body at the second end includes a first discrete end and a second discrete end;
the second aperture is located adjacent the first discrete end of the second end;
a third aperture is located in the body adjacent the second discrete end of the second end;
the second leg extends from the body interiorly adjacent of the second aperture at the first discrete end of the second end; and
a third second leg extends from the body interiorly adjacent of the third aperture at the second discrete end of the second end.

21. The orthopedic implant according to claim 1, wherein:
the body at the first end includes a first discrete end and a second discrete end;
the body at the second end includes a first discrete end and a second discrete end;
the first aperture is located adjacent the first discrete end of the first end;
the second aperture is located adjacent the first discrete end of the second end;
a third aperture is located in the body adjacent the second discrete end of the first end;
a fourth aperture is located in the body adjacent the second discrete end of the second end;
the first leg extends from the body interiorly adjacent of the first aperture at the first discrete end of the first end;
the second leg extends from the body interiorly adjacent of the second aperture at the first discrete end of the second end;
a third second leg extends from the body interiorly adjacent of the third aperture at the second discrete end of the first end; and
a fourth leg extends from the body interiorly adjacent of the fourth aperture at the second discrete end of the second end.

22. A method for an orthopedic implant, comprising:
providing an orthopedic implant, comprising:
a body deformable between an unconstrained form and a constrained insertion form that transitions the orthopedic implant between an unconstrained shape and a constrained insertion shape, and
first and second legs extending from the body whereby deformation of the body moves the first and second legs between an unconstrained position and a constrained insertion position;
deforming the body from its unconstrained form to its constrained insertion form causing the first and second legs to move from to their unconstrained position to their constrained insertion, thereby transitioning the orthopedic implant from its unconstrained shape to its constrained insertion shape;
retaining the orthopedic implant in its constrained insertion shape;
implanting the orthopedic implant in a bone or tissue;
releasing the orthopedic implant whereby the body attempts to transition from its constrained insertion form to its unconstrained form resulting in the first and second legs attempting to move from their constrained insertion position to their unconstrained position, thereby exerting a compressive force to the bone or tissue that holds the bone or tissue in a desired alignment;
securing a first end of the body with the bone or tissue using a first fixation device;
securing a second end of the body with the bone or tissue using a second fixation device whereby the first and second legs and the first and second fixation devices are aligned lengthwise along the body such that the first and second legs are spaced apart at a first distance and the first and second fixation devices are spaced apart at a second distance that is greater than the first distance.
